(19) United States
(12) Patent Application Publication
(10) Pub. No.: US 2023/0317247 A1
(43) Pub. Date: Oct. 5, 2023

(54) HEALTH CARE DEVICE AND HEALTH CARE METHOD

(71) Applicant: TAIWAN OASIS TECHNOLOGY CO., LTD., NEW TAIPEI CITY (TW)

(72) Inventors: MING-SHUN LEE, NEW TAIPEI CITY (TW); CHIN-SUNG TSENG, NEW TAIPEI CITY (TW); HSU-HUI TSENG, NEW TAIPEI CITY (TW); HSIEN-CHING TSENG, NEW TAIPEI CITY (TW); WEI-LONG LEE, NEW TAIPEI CITY (TW)

(21) Appl. No.: 17/745,162

(22) Filed: May 16, 2022

(30) Foreign Application Priority Data
Mar. 31, 2022 (TW) 111112702

(51) Int. Cl. G16H 20/90

(52) U.S. Cl. CPC G16H 20/90

(57) ABSTRACT

A health care device and health care method are illustrated. A health care device is used to emit a low-frequency wave of a predetermined frequency forward to an umbilicus of a user, wherein the low-frequency wave is emitted at a predetermined position, and the predetermined position is located in front of the umbilicus of the user with a predetermined distance. The health care device comprises the low-frequency wave emitter, a column and a cone. The predetermined frequency is within a range between 1.27 Hz and 1.81 Hz, and the predetermined distance D is within a range between 5 cm and 8 cm. The health care device and the health care method can increase the content of activated T cells in the blood, so as to achieve effects of convenient operation, accurate identification of the use position, non-invasive treatment, non-contact treatment, and no harm caused by excessive energy risk.

### BACKGROUND

### Technical Field

The present disclosure relates to a health care device and a health care method, and particularly to a health care device and a health care method, each of which emits a low-frequency wave to a human body for health care.

### Related Art

Known health care at present for example comprises scraping, cupping, acupuncture, massage etc., although the purpose of health care can be achieved, there are still shortcomings. For example, scraping may make skin damaged, and increases the risk of infection. Effect of cupping is similar to that of scraping, but the use of the can requires contact with the skin, so the cleanliness and hygiene of the can is a concern. Acupuncture uses needles to penetrate the corresponding acupoints of the human body, so there is an invasive risk. Although massage has no invasive risk, it relies on others to help relax the muscles.

The health care of acupuncture is improved by others, and a microwave acupuncture instrument which adopts the health care device emitting non-invasive radiation waves to human acupoints is proposed. An oscillation frequency of the radiation waves emitted by the microwave oscillator in the microwave acupuncture instrument is 1000 MHz to 2000 MHz. The oscillation frequency is very high, so that it is very easy to cause damage due to excessive radiation (energy). In practice, to achieve the desired effect of health care, the propagating direction of radiation wave of the microwave acupuncture instrument also needs to align to corresponding acupoints of the human body. However, most people are not professionals related to traditional Chinese medicine and cannot accurately identify each person's individual acupoints. Therefore, the microwave acupuncture instrument is not actually a universal health care device.

Therefore, it is a trend in the industry to develop a health care device and a health care method that can achieve effects of convenient operation, accurate identification of the use position, non-invasive treatment, non-contact treatment, and no harm caused by excessive energy risk.

### SUMMARY

The present disclosure is mainly based on recognizing that the main tissues and organs of the human body (referring to organisms, rather than artificial tissues or organs) have inherent oscillation frequencies, such as 8-12 Hz for the head, 4-6 Hz for the thorax, and 5 Hz for the heart, 6-9 Hz for the abdominal cavity, 1.2 Hz for the blood flow, and 1.8 Hz for the bone tissue. When a certain part of the human body is in the low-frequency wave field of 0-20 Hz, such as a infrasound wave or infrasound (by contrast, the sound is called the ultrasonic or ultrasonic waves if the frequency is higher than 20 KHz), if its sound pressure level (the unit is dB) reaches a certain threshold, the elastic wall of the outer layer of the tissue and organ corresponding to this part will first enter the state of vibration, and then the vibration will be transmitted to the interior of the tissue and organ. In particular, resonance occurs when the frequency of the infrasound wave is synchronized with the aforementioned inherent oscillation frequency of the tissues and organs, and the tissues and organs receive the greatest stimulation at this time. When the resonance caused by infrasound waves acts on tissues and organs, it also acts on the cellular structures of tissues and organs, such as the mitochondrion of cells, which is the main place of the oxidative phosphorylation and synthesis of adenosine triphosphate (ATP) in cells. The oxidative phosphorylation and synthesis of adenosine triphosphate (ATP) in cells can provide chemical energy for cell activities, so the mitochondrion is called the powerhouse of the cell. In addition, the resonance induced by the aforementioned infrasound wave acts on the mitochondria and also affects the binding state of some enzymes to the cell membrane and the activity of the enzymes. At the same time, when the infrasound wave of a certain intensity acts, the energy of the wave can be converted into heat energy, biochemical energy and bioelectric energy, which can directly act on the tissues and organs, so that the damaged tissues can be repaired and restored to normal under the stimulation of the infrasound wave. Using the infrasound wave as the component of the health care device can avoid the problem of injury caused by the excessive energy risk generated by the high frequency microwave oscillator. In addition, it is particularly noted here that the above infrasound wave is only an example of a low-frequency wave, and a low-frequency wave may also include electromagnetic waves such as radio waves or light waves.

In the present disclosure, the theory of traditional Chinese medicine is recognized, the umbilicus of the human body is the Shenque acupoint, and is the place where Ren meridian, Du meridian, Dai meridian, Chong meridian pass through. Thus, the whole body can be adjusted via the meridian which contacts internal organs when stimulating Shenque acupoint. Furthermore, according to the theory of modern medicine, the umbilicus is the thinnest part of the epidermis stratum corneum in the abdomen of the human body and there is no adipose tissue under the umbilicus, so its barrier function is the weakest. In addition, the umbilicus skin has a rich venous network and inferior abdominal artery branches in addition to the microcirculation blood vessels of the general skin. Therefore, as long as some effect or change of the blood in the microcirculation blood vessels of the umbilicus skin is performed, the effect or change is brought into the human blood circulatory system. The umbilicus of the human is easy to identify, and its position will not vary depending on the person's height, weight and body shape. Therefore, using umbilicus as the corresponding position of the human body which a health care device to apply will have the advantage of allowing users to accurately identify the use position.

In the present disclosure, the infrasound wave emitter is not in contact with the umbilicus, and the infrasound wave emitter is made to launch the infrasound wave towards the umbilicus to achieve the effect of the acupuncture, so that non-invasive, non-contact health care can be achieved. Since it is recognized that the infrasound wave can achieve the effect similar to that of acupuncture, and the effect of acupuncture on the immune system is that acupuncture can increase the content of interferon gamma (γ-IFN) and neuropeptide beta-endorphin (β endorphin), so as to activate NK cells (natural killer cells), wherein the NK cells secrete cytokines and regulate the inflammatory response. The NK cells are immune cells with large granules in the cytoplasm, developed from bone marrow lymphoid stem cells, mainly distributed in peripheral blood (including blood in microcirculation blood vessels) and spleen. There also a small presence of the NK cells in lymph nodes and other organization.

As mentioned above, the umbilicus is the thinnest part of the epidermis stratum corneum in the abdomen of the human body and there is no adipose tissue under the umbilicus, so its barrier function is the weakest, and the umbilicus skin has a rich venous network and inferior abdominal artery branches in addition to the microcirculation blood vessels of the general skin, so that as long as some effect or change of the blood in the microcirculation blood vessels of the umbilicus skin is performed, the effect or change is brought into the human blood circulatory system. Therefore, when the infrasound wave emitter emits the infrasound wave towards the umbilicus, it will trigger an effect similar to that of the acupuncture, which will increase the content of γ-IFN and β endorphins in the blood of the umbilicus skin microcirculation blood vessels and activate NK cells, B cells (B lymphocytes) and active T cells (active T lymphocytes), and because NK cells, B cells and active T cells are activated, they secrete cytokines, therefore regulating inflammation or secreting antibodies. To sum up, the effect of the health care device of the present disclosure can be proofed as long as the changes in the content of NK cells, B cells or active T cells in the blood are detected.

Based upon the above explanations, the present disclosure provides a health care method. A low-frequency wave emitter is used to emit a low-frequency wave of a predetermined frequency forward to an umbilicus of a user, wherein the low-frequency wave is emitted at a predetermined position, and the predetermined position is located in front of the umbilicus of the user with a predetermined distance.

Based upon the above explanations, the present disclosure provides another one health care method. A health care device is used to emit a low-frequency wave of a predetermined frequency forward to an umbilicus of a user, wherein the low-frequency wave is emitted at a predetermined position, and the predetermined position is located in front of the umbilicus of the user with a predetermined distance. The health care device comprises a low-frequency wave emitter, a column and a cone. The column has top surface, a bottom surface and a column body. The two ends of the column body are respectively connected to the top surface of the column and the bottom surface of the column. The cone has a vertex, a bottom surface and a cone body, and the two ends of the cone body are respectively connected to the vertex and the bottom surface of the cone. The top surface of the column is correspondingly jointed to the bottom surface of the cone, and the low-frequency wave emitter is connected to the bottom surface of the column.

In an embodiment, the predetermined position is located on a virtual bottom surface of a virtual cone, a virtual vertex of the virtual cone is the umbilicus, and the predetermined distance is within a range between 5 cm and 8 cm. The predetermined position is located on a center of the virtual bottom surface, the virtual cone is a circular cone, and the predetermined position is located on a circle center of the virtual bottom surface being a circle, the predetermined distance is a distance between the circle center and the umbilicus, and the vertex is located at the predetermined position.

In an embodiment, the vertex further has a stick being vertically standing and having a longitudinal direction away from the bottom surface of the cone, the longitudinal direction of the stick is coincided with a specific normal line of the bottom surface of the cone and extending towards the umbilicus, the specific normal line of the bottom surface of the cone passes through a bottom surface center of the cone, and a stick tip of the stick is located at the predetermined position.

Based upon the above explanations, the present disclosure provides a health care device. The health care device comprises a low-frequency wave emitter, a column and a cone. The column has top surface, a bottom surface and a column body. The two ends of the column body are respectively connected to the top surface of the column and the bottom surface of the column. The cone has a vertex, a bottom surface and a cone body, and the two ends of the cone body are respectively connected to the vertex and the bottom surface of the cone. The top surface of the column is correspondingly jointed to the bottom surface of the cone, and the low-frequency wave emitter is connected to the bottom surface of the column. The low-frequency wave emitter is used to emit a low-frequency wave of a predetermined frequency.

In an embodiment, the predetermined frequency is within a range between 1.27 Hz and 1.8 Hz.

In an embodiment, the predetermined frequency is 1.45 Hz.

To sum up, the health care device and the health care method provide by the present disclosure can achieve effects of convenient operation, accurate identification of the use position, non-invasive treatment, non-contact treatment, and no harm caused by excessive energy risk.

### BRIEF DESCRIPTION OF THE DRAWINGS

The accompanying drawings are included to provide a further understanding of the present disclosure, and are incorporated in and constitute a part of this specification. The drawings illustrate exemplary embodiments of the present disclosure and, together with the description, serve to explain the principles of the present disclosure.

FIG. 1 is a schematic diagram of executing a health care method according to an embodiment of the present disclosure.

FIG. 2 is a schematic diagram showing a structure of a health care device when executing a health care method according to an embodiment of the present disclosure.

FIG. 3 is a schematic diagram showing a structure of a health care device according to an embodiment of the present disclosure.

FIG. 4 is a schematic diagram showing a structure of a health care device with a tube according to an embodiment of the present disclosure.

FIG. 5 is a schematic diagram showing a structure of a health care device with a vertically standing stick according to an embodiment of the present disclosure.

FIG. 6 is a schematic diagram showing a structure of a health care device installed on or in a frame according to an embodiment of the present disclosure.

### DESCRIPTION OF THE EXEMPLARY EMBODIMENTS

To understand the technical features, content and advantages of the present disclosure and its efficacy, the present disclosure will be described in detail with reference to the accompanying drawings. The drawings are for illustrative and auxiliary purposes only and may not necessarily be the true scale and precise configuration of the present disclosure. Therefore, the scope of the present disclosure should not be limited to the scale and configuration of the attached drawings.

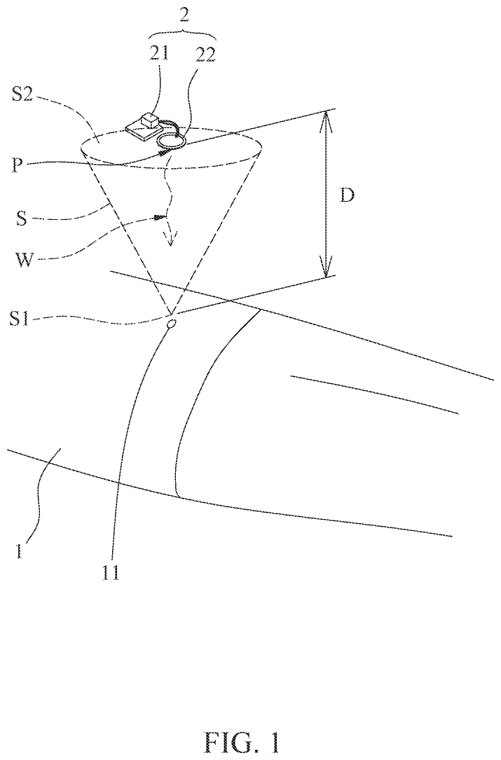

Refer to FIG. 1, the present disclosure provides a health care method. The method uses a low-frequency wave emitter 2 to emit a low-frequency wave W of a predetermined frequency forward to an umbilicus 11 of a user 1, wherein the low-frequency wave W is emitted at a predetermined position P, and the predetermined position P is located in front of the umbilicus 11 of the user 1 with a predetermined distance D. The aforementioned “front” refers to the front of the body in the direction of the abdomen, and correspondingly, the “back” refers to the back of the human body. The predetermined position P is located on a virtual bottom surface S2 of a virtual cone S with the umbilicus 11 as a virtual vertex S1 of the virtual cone S. The predetermined distance D is designed to be not larger than 8 cm, or the predetermined distance D is designed to be not less than 5 cm, or the predetermined distance D is designed to be within a range between 5 cm and 8 cm (p.s. the range comprises two end values of 5 cm and 8 cm). Preferably, the predetermined position P is located on the center of the virtual bottom surface S2, for example, the virtual cone S is a circle cone, the predetermined position P is located on the circle center of the virtual bottom surface S2 being circle, and the predetermined distance D is the distance between circle center and the umbilicus 11, which is within the range between 5 cm and 8 cm.

The low-frequency wave emitter 2 is preferably a infrasound wave transducer, and the low-frequency wave W can be a infrasound wave. The structure of the infrasound wave transducer comprises a power 21 and a vibration part 22. The vibration part 22 is electrically connected to the power 21. The vibration part 22 is a disc-shaped piezoelectric ceramic transducer. The piezoelectric ceramic transducer is made of piezoelectric material polarized in the thickness direction. The power 21 provides power to the vibration part 22 to make the vibration part 22 vibrates in the thickness direction to generate the infrasound wave of the predetermined frequency. In the present disclosure, the predetermined frequency is not larger than 1.81 Hz, or the predetermined frequency is not less than 1.27 Hz, or the predetermined frequency is within a range between 1.27 Hz and 1.81 Hz (p.s. the range comprises two end values of 1.27 Hz and 1.8 Hz). Preferably, the predetermined frequency is 1.45 Hz.

<Detection Manner and Effect>

The health care method is executed to personnel P and personnel Q, the predetermined distance D is selected to be 5 cm, the predetermined frequency is selected to be 1.45 Hz, and the low-frequency wave is selected to be the infrasound wave. The personnel P is a 74 years old man, the personnel Q is a 72 years old woman. Before executing the aforementioned health care method, blood samples are drawn from the personnel P and Q and sent to the medical laboratory for immunological examination to check the percentage (i.e. content) of active T cells. On the 19-th day after the execution of the aforementioned health care method for 2 hours, the blood sample of the personnel P is again taken and sent to the medical laboratory for immunological examination, and on the 42-nd day after the execution of the aforementioned health care method for 2 hours, the blood sample of the personnel Q is also taken again and sent to the medical laboratory for immunological examination. The percentages of active T cells of the blood samples are examined in the same way, and the results are recorded in TABLE 1 which is described later.

TABLE 1

| | PERSONNEL P | | | PERSONNEL Q | | |
|---|---|---|---|---|---|---|
| | | | CHANGE RATE = | | | CHANGE RATE = |
| | | | CONTENT OF ACTIVE | | | CONTENT OF ACTIVE |
| | | | T CELLS AFTER | | | T CELLS AFTER |
| | | | EXECUTION/CONTENT | | | EXECUTION/CONTENT |
| | BEFORE | AFTER | OF ACTIVE T CELLS | BEFORE | AFTER | OF ACTIVE T CELLS |
| | EXECUTION | EXECUTION | BEFORE EXECUTION | EXECUTION | EXECUTION | BEFORE EXECUTION |
| CONTENT | 6.2% | 12.8% | 206.5% | 5.3% | 7.9% | 149% |
| OF ACTIVE | | | | | | |
| T CELLS | | | | | | |

From TABLE 1, before executing the aforementioned health care method, the content of active T cells in personnel P is 6.2%, and after executing the aforementioned health care method, the content of active T cells increased to 12.8%, and the change rate is 206.5%, that is, the content of active T cells is increased 106.5%. Before executing the aforementioned health care method, the content of active T cells in personnel Q is 5.3%, and after executing the aforementioned health care method, the content of active T cells is increased to 7.9%, and the change rate is 149%, that is, the content of active T cells is increased 49%. Therefore, the health care method provided by the present disclosure can promote the proliferation of active T cells, and as long as a single execution of 2 hours can make the content of active T cells increase, even after 42 days, still can maintain 49% increase quantity. Apparently, the health care method of the present disclosure is highly effective and can maintain long-term health effects.

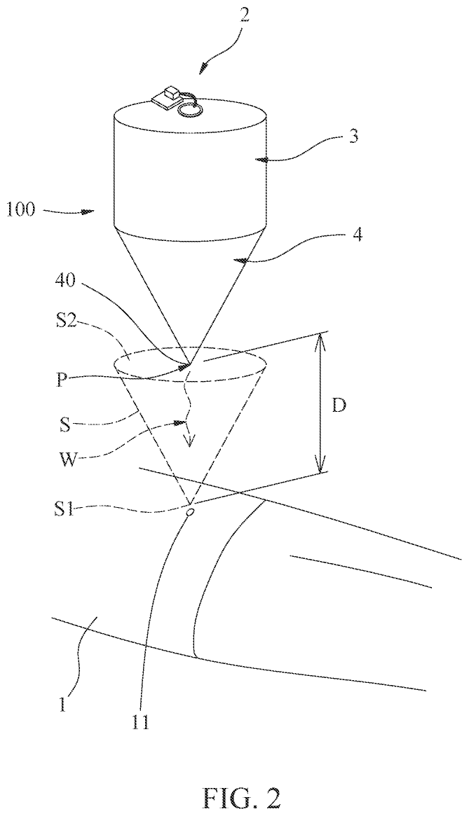

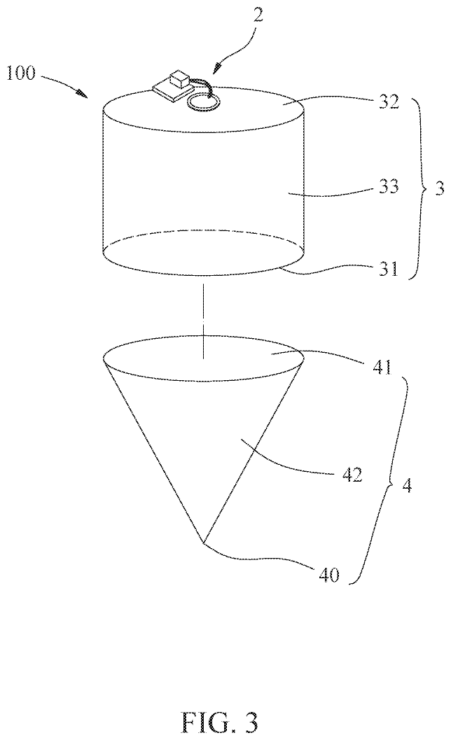

Refer to FIG. 2 and FIG. 3, the present disclosure further provides a health care device 100 for executing the above health care method. The health care device 100 comprises the low-frequency wave emitter 2, a column 3 and a cone 4. The column 3 is preferably the circle column, the column 3 has a top surface 31, a bottom surface 32 and a column body 33, and the two ends of the column body 33 are respectively connected to the top surface 31 of column 3 and the bottom surface 32 of column 3. The cone 4 has a vertex 40, a bottom surface 41 and a cone body 42, wherein two end of the cone body 42 are respectively connected to the vertex 40 and the bottom surface 41 of the cone 4. The top surface 31 of the column 3 is correspondingly joined with the bottom surface 41 of the cone 4, for example, by welding or screwing. Preferably, the column 3 and the cone 4 are metal columns and metal cones, respectively. More preferably, the column 3 and the cone 4 are formed integrally, in other words, the top surface 31 of the column 3, the column body 33, the cone body 42 and the vertex 40 are formed integrally.

The low-frequency wave emitter 2 is connected with the bottom surface 32 of the column 3, preferably this low-frequency wave emitter 2 is directly connected with the bottom surface 32 of the column 3, such as this low-frequency wave emitter 2 is welded or screwed to the bottom surface 32 of the column 3, so when the power 21 is activated, the vibration part 22 will generate the low-frequency wave W of the predetermined frequency (please refer to FIG. 1 again). Next, the low-frequency wave W is transmitted to this bottom surface of column 32, and the column body 33 is used to transmit the low-frequency wave W sequentially to the bottom surface 41 of the cone 4 and this vertex 40. By using the structure of the cone 4 which the bottom surface 41 of the cone 4 is designed to be wider than and tapered to the vertex 40, the energy of the low-frequency wave W is more concentrated as it is transmitted towards the vertex 40. Therefore, when the low-frequency wave W is delivered to this vertex 40, the energy of the low-frequency wave W is gathered to the maximum, and then emitted towards the umbilicus 11 from the vertex 40. The vertex 40 of the cone 4 is a tip, and therefore, based on the principle of the tip discharge in electricity, the low-frequency wave W can be concentrated on the vertex 40 to be released and emitted towards the umbilicus 11. It is noted that, the vertex 40 is located at the predetermined position P at this time.

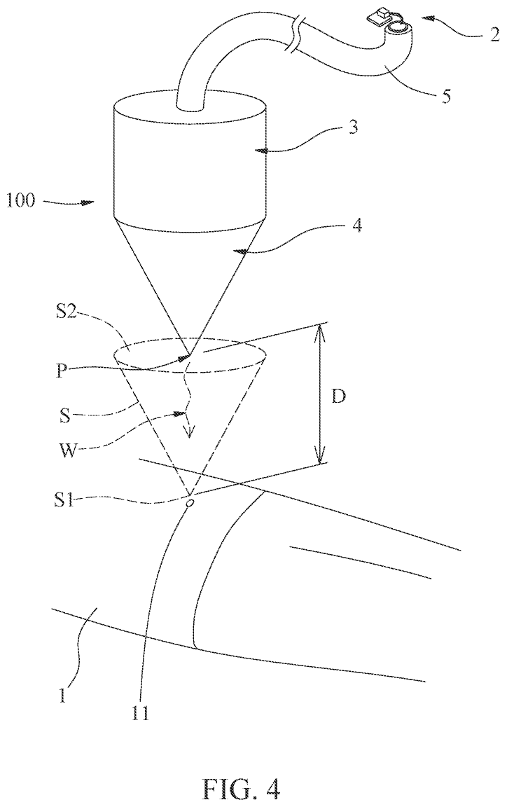

Refer to FIG. 4, and in another one embodiment, the low-frequency wave emitter 2 is connected to the bottom surface of column 32 via a tube 5. The low-frequency wave W is transmitted to the bottom surface 32 of the column via the tube 5. The low-frequency wave W then passes to the vertex 40 and is emitted towards the umbilicus 11 as previously described. With the arrangement of the tube 5, the power 21 and/or the vibration part 22 of the low-frequency wave emitter 2 can be kept away from the human body, so as to avoid interference with the electronic device implanted in the human body.

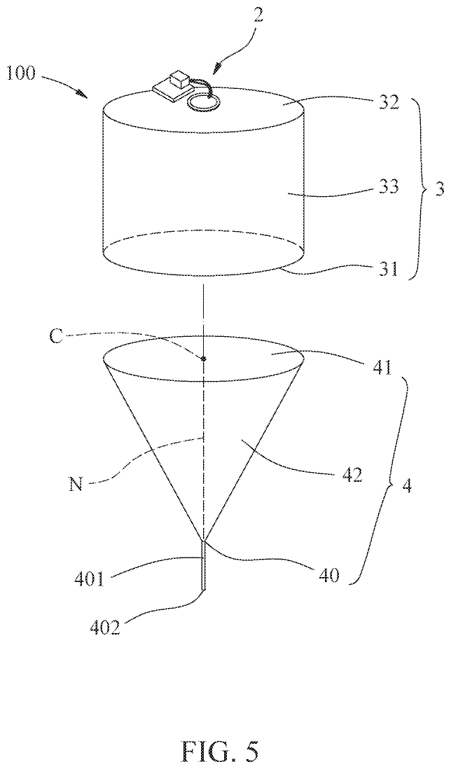

Refer to FIG. 5, and in another one embodiment, the vertex 40 further has a stick 401 being vertically standing and having a longitudinal direction away from the bottom surface 41 of the cone 4. Preferably, the stick 401 is a metal stick such as metal silk or metal wire. The longitudinal direction of the stick 401 is coincided with a specific normal line N of the bottom surface 41 of the cone 4, and the specific normal line N of the bottom surface 41 of the cone 4 passes through a bottom surface center C of the cone 4 and extends towards the umbilicus 11. Therefore, the energy of the low-frequency wave W can be more directed to concentrate on a stick tip 402 of the stick 401 to be released and emitted towards the umbilicus 11. Specifically, the stick tip 402 is located at the predetermined position P at this time.

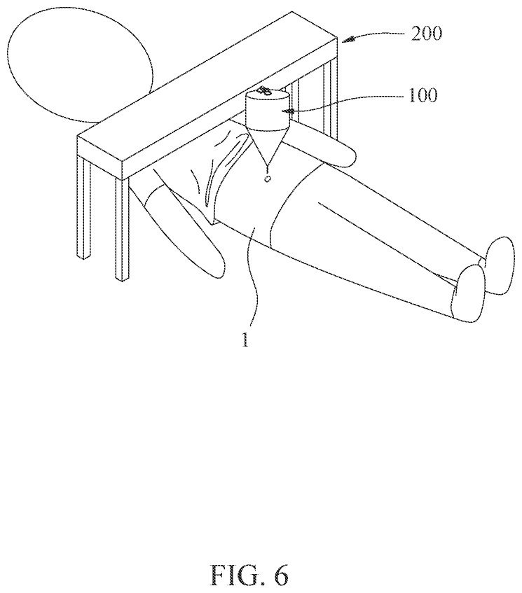

Refer to FIG. 6, in another one embodiment, the health care device 100 is installed in or on a frame 200, the user 1 is lying on the platform (not shown in the drawings), the frame 200 is erected on the platform, and the health care device 100 is located above the umbilicus of the user 1. In this way, the user 1 can also get a proper rest during the health care process.

The above-mentioned descriptions represent merely the exemplary embodiment of the present disclosure, without any intention to limit the scope of the present disclosure thereto. Various equivalent changes, alternations or modifications based on the claims of present disclosure are all consequently viewed as being embraced by the scope of the present disclosure.

We claim:

1. A health care device, comprising:
a low-frequency wave emitter, configured to emit a low-frequency wave of a predetermined frequency;
a column, having a top surface, a bottom surface and a column body which has two ends respectively connected to the top surface of the column and the bottom surface of the column; and
a cone, having a vertex, a bottom surface and a cone body which has two ends respectively connected to the vertex and the bottom surface of the cone;
wherein the top surface of the column is correspondingly jointed to the bottom surface of the cone, and the low-frequency wave emitter is connected to the bottom surface of column.

2. The health care device according to claim 1, wherein the predetermined frequency is within a range between 1.27 Hz and 1.8 Hz.

3. The health care device according to claim 2, wherein the predetermined frequency is 1.45 Hz.

4. The health care device according to claim 3, wherein the low-frequency wave emitter is a infrasound wave transducer, and the low-frequency wave is a infrasound wave.

5. The health care device according to claim 4, wherein the column is a cylinder, and the column and the cone are formed integrally.

6. The health care device according to claim 5, wherein the low-frequency wave emitter is connected to the bottom surface of the column via a tube.

7. The health care device according to claim 6, wherein the vertex further has a stick being vertically standing and having a longitudinal direction away from the bottom surface of the cone.

8. The health care device according to claim 7, wherein the longitudinal direction of the stick is coincided with a specific normal line of the bottom surface of the cone, and the specific normal line of the bottom surface of the cone passes through a bottom surface center of the cone.

9. A health care method, comprising:
using a low-frequency wave emitter to emit a low-frequency wave of a predetermined frequency forward to an umbilicus of a user, wherein the low-frequency wave is emitted at a predetermined position, and the predetermined position is located in front of the umbilicus of the user with a predetermined distance.

10. The health care method according to claim 9, wherein the predetermined frequency is within a range between 1.27 Hz and 1.8 Hz.

11. The health care method according to claim 10, wherein the predetermined frequency is 1.45 Hz.

12. The health care method according to claim 11, wherein the low-frequency wave emitter is a infrasound wave transducer, and the low-frequency wave is a infrasound wave.

13. The health care method according to claim 12, wherein the predetermined position is located on a virtual bottom surface of a virtual cone, a virtual vertex of the virtual cone is the umbilicus, and the predetermined distance is within a range between 5 cm and 8 cm.

14. The health care method according to claim 13, wherein the predetermined position is located on a center of the virtual bottom surface.

15. The health care method according to claim 14, wherein the virtual cone is a circular cone, and the predetermined position is located on a circle center of the virtual bottom surface being a circle, and the predetermined distance is a distance between the circle center and the umbilicus.

16. A health care method, comprising:
using a health care device to emit a low-frequency wave of a predetermined frequency forward to an umbilicus of a user, wherein the low-frequency wave is emitted at a predetermined position, and the predetermined position is located in front of the umbilicus of the user with a predetermined distance;
wherein the health care device comprises:
a low-frequency wave emitter, configured to emit a low-frequency wave of a predetermined frequency;
a column, having a top surface, a bottom surface and a column body which has two ends respectively connected to the top surface of the column and the bottom surface of the column; and
a cone, having a vertex, a bottom surface and a cone body which has two ends respectively connected to the vertex and the bottom surface of the cone;
wherein the top surface of the column is correspondingly jointed to the bottom surface of the cone, and the low-frequency wave emitter is connected to the bottom surface of column.

17. The health care method according to claim 16, wherein the predetermined frequency is within a range between 1.27 Hz and 1.8 Hz.

18. The health care method according to claim 17, wherein the predetermined frequency is 1.45 Hz.

19. The health care method according to claim 18, wherein the low-frequency wave emitter is a infrasound wave transducer, and the low-frequency wave is a infrasound wave.

20. The health care method according to claim 19, wherein the predetermined position is located on a virtual bottom surface of a virtual cone, a virtual vertex of the virtual cone is the umbilicus, and the predetermined distance is within a range between 5 cm and 8 cm; the predetermined position is located on a center of the virtual bottom surface, the virtual cone is a circular cone, and the predetermined position is located on a circle center of the virtual bottom surface being a circle, the predetermined distance is a distance between the circle center and the umbilicus, and the vertex is located at the predetermined position.

21. The health care method according to claim 19, wherein the vertex further has a stick being vertically standing and having a longitudinal direction away from the bottom surface of the cone, the longitudinal direction of the stick is coincided with a specific normal line of the bottom surface of the cone and extending towards the umbilicus, the specific normal line of the bottom surface of the cone passes through a bottom surface center of the cone, and a stick tip of the stick is located at the predetermined position.
